(19) United States
(12) Patent Application Publication
(10) Pub. No.: US 2025/0264447 A1
(43) Pub. Date: Aug. 21, 2025

(54) GAS MEASURING SYSTEM FOR DETERMINING A QUALITY PARAMETER OF SEED STOCK

(71) Applicant: Dräger Safety AG & Co. KGaA, Lübeck (DE)

(72) Inventors: Michael Sick, Lübeck (DE); Marie-Isabell Mattern-Frühwald, Lübeck (DE); Silja Dennier, Lübeck (DE); Steffen Rittemann, Lübeck (DE)

(21) Appl. No.: 19/053,976

(22) Filed: Feb. 14, 2025

(30) Foreign Application Priority Data
Feb. 15, 2024 (DE) 102024104274.5

(51) Int. Cl. G01N 33/00

(52) U.S. Cl. CPC G01N 33/0027; G01N 33/0098

(57) ABSTRACT

The disclosure provides a gas measuring system for determining a quality parameter of seed stock. The gas measuring system may include a sample chamber for receiving a seed of the seed stock; a number of electrochemical gas sensors which are fluidically connected to the sample chamber in order to determine a number of concentrations of a number of target components of a gas present in the sample chamber and to provide measurement signals corresponding to the number of concentrations, wherein the determination of the number of concentrations and the provision of the measurement signals take place continuously; and an evaluation unit which is configured to receive the measurement signals automatically, to determine the quality parameter from the measurement signals, and to provide the quality parameter via a data interface.

### CROSS-REFERENCE TO RELATED APPLICATIONS

This application claims the priority of German Patent Application No. 102024104274.5, filed on Feb. 15, 2024, and titled “GAS MEASURING SYSTEM FOR DETERMINING A QUALITY PARAMETER OF SEED STOCK”, which is hereby incorporated by reference in its entirety for all nonlimiting purposes.

### SUMMARY

The present disclosure relates to a gas measuring system for determining a quality parameter of seed stock.

One goal in the commercial use of seed stock is to use a seed stock of the highest possible quality in order to achieve a high yield. This creates the need to be able to make a statement about the quality of seed stock at different stages of seed stock production.

In commercial seed stock production, the quality of seed stock is determined by means of complex, predominantly manual methods. Seedlings are grown individually into plants, and an assessment of their germination capacity is made using a sample size of at least 100 plants. The assessment is based upon predominantly subjective, visual criteria.

Other examination methods are known, such as a tetrazolium test (color test of the vital seeds), an on-farm vigor check with reactive samples in which the color changes in the presence of ethanol, and complex X-ray examinations of the seeds.

There are efforts in research to develop new methods. In this respect, the publication Buckley, W. et al., Canola Seed Vigour Ethanol Test, 2003, pp. 150-156, hereby incorporated by reference, mentions a method in which a large number of seeds are brought to germination in a common container, from which an ethanol sample is taken once after a predetermined time by means of a gas measuring device that is not optimized for this purpose. From these experiments, it is known that a quantity of ethanol produced during the germination process is directly related to the quality of the seeds.

Without being bound to this theory, this disclosure assumes that, during the onset of germination of a seed of good quality, anaerobic energy production takes place, which is associated with the breakdown of glucose into lactose and ethanol. After a while, the good seed then converts to an aerobic metabolism, in which oxygen from the environment is consumed in a downstream reaction, and ethanol is no longer formed. On the other hand, in the case of damaged, treated, or immature seeds, anaerobic energy production continues, and, since no conversion takes place, ethanol continues to be produced.

However, this method has been shown to have low accuracy in determining the germination capacity and to require manual interaction with the seeds.

It is therefore an object of the present disclosure to provide a gas measuring system for determining a quality parameter of seed stock, with which an accurate statement about the quality of a seed stock sample can be made in a simple, reproducible manner.

These and other objects are achieved by the subject matter of the present disclosure.

The claims, the description, and the figures provide advantageous embodiments of the present disclosure.

The gas measuring system according to the disclosure for determining a quality parameter of seed stock has a sample chamber for receiving at least (preferably, exactly) one seed of the seed stock; a number of electrochemical gas sensors which are fluidically connected to the sample chamber in order to determine a number of concentrations of a number of target components of a gas present in the sample chamber and to provide measurement signals corresponding to the number of concentrations, wherein the determination of the number of concentrations and the provision of the measurement signals take place continuously; and an evaluation unit which is configured to receive the measurement signals automatically, to determine the quality parameter from the measurement signals, and to provide the quality parameter via a data interface.

In this way, a gas measuring system can be provided which makes it possible to provide the quality parameter automatically and objectively.

A particular advantage over previously known methods for determining quality parameters of seed stock is, on the one hand, that continuous determination of the number of concentrations and provision of the measurement signals take place, which is not possible with only a single measurement of an ethanol concentration. This disclosure makes it possible to ascertain a time-resolved course of the concentration of the number of target components and thus to obtain more and better information about the germination process and include it in the determination of the quality parameter. In the context of the disclosure, it was recognized that the quality of seed stock and thus also the quality parameter is indicated or influenced not only by the total quantity of the number of target components formed or consumed during a measurement period, but also in particular by the temporal course of the number of target components.

On the other hand, the gas measuring system according to the disclosure makes it possible to carry out a single seed measurement (also referred to as a single grain measurement) or a plurality of respective single seed measurements in parallel. As a result, a statement about the quality of each seed in a quantity of seeds can be provided.

Overall, a gas measuring system that makes detailed monitoring of the entire germination phase of a seed possible can thus be provided. In addition, it is possible to examine, at the level of individual seeds, what differences and similarities exist between the properties of the individual seeds in a quantity of seeds.

For improving the handling and the measurement environment, it is preferred that the sample chamber be formed by a sample container whose interior delimiting the sample chamber has a volume which is adapted to the type of seed stock to be used therewith, in such a way that a concentration of the target components influenced (i.e., formed and/or consumed) during germination in the gas composition initially present in the sample chamber is in a range measurable by the number of electrochemical gas sensors.

It is possible to determine and provide more than one quality parameter by means of the gas measuring system according to the disclosure. For example, two quality parameters can be determined and provided. However, the provision of only one quality parameter is preferred. It is also possible to combine a plurality of quality parameters (e.g., in a weighted manner) in order to provide a combined quality parameter as a quality parameter.

The gas measuring system according to the disclosure can be designed as a compact assembly or can be distributed locally (e.g., as a multi-sensor array).

For example, the number of electrochemical gas sensors can be arranged on the sample chamber and can be directly fluidically connected thereto. Alternatively, the number of electrochemical gas sensors can be arranged locally separated from the sample chamber and can be indirectly fluidically connected thereto via a line.

The evaluation unit can, for example, be designed as an electrical or electronic circuit and/or as a microprocessor on a circuit board. If the evaluation unit is designed as a circuit board, it is preferred that it be accommodated on one or all of the number of electrochemical gas sensors.

For example, the evaluation unit can be designed additionally or alternatively as a data processing system, such as a PC or as a handheld device with suitable programming. If the evaluation unit is designed as a data processing system, it is preferred that it be provided locally separated from the number of electrochemical gas sensors and be or be able to be connected thereto wirelessly or by cable.

The gas measuring system may (as part of the evaluation unit or as a separate component of the gas measuring system) comprise a potentiostat or have a circuit or programming that functions as a potentiostat.

The target component can be a gaseous component of the gas present in the sample chamber or formed during germination.

It is possible to provide exactly one electrochemical gas sensor or a plurality of electrochemical gas sensors as the number of electrochemical gas sensors.

Each of the number of electrochemical gas sensors or only a portion of the number of electrochemical gas sensors can be configured to measure exactly one target component or to measure a plurality of target components, preferably simultaneously.

Each sample chamber can be fluidically connected to exactly one electrochemical gas sensor or to a plurality of electrochemical gas sensors.

Each of the number of electrochemical gas sensors can be designed as a simple sensor or as a multisensor—for example, as a double sensor or as a triple sensor. In this respect, it is possible for an electrochemical gas sensor, some electrochemical gas sensors, or all electrochemical gas sensors of the number of electrochemical gas sensors to provide exactly one measurement signal if it is or they are designed as simple sensors, or to provide a plurality of measurement signals if it is or they are designed as multisensors.

It is preferred that each sample chamber be fluidically connected to a plurality of simple sensors and/or to at least one multisensor, wherein the plurality of simple sensors and/or the multisensor are configured to measure oxygen and ethanol as target components.

Preferably, the number of electrochemical gas sensors are configured to determine a concentration of ethanol as a target component of the number of target components. Additionally or alternatively, it is preferred that the number of electrochemical gas sensors be configured to determine a concentration of oxygen and/or a concentration of carbon dioxide as target components of the number of target components.

It has been described above that a statement about the germination capacity of seed stock can be made at least by considering the temporal course of an ethanol concentration of the gas present in the sample chamber or a quantity of ethanol that can be determined from this value. In the context of the disclosure, it was further recognized that additionally or alternatively determining oxygen and/or carbon dioxide as target components allows the determination of the quality parameter to be even better adapted to the behavior of the seed stock to be examined.

Thus, without being bound to this theory, this disclosure assumes that there may be a correlation between the temporal development of an oxygen concentration and/or an oxygen quantity and/or a carbon dioxide concentration and/or a carbon dioxide quantity and the germination state of the seed, which correlation can be included in the determination of the quality parameter or taken into account in the determination of one or more additional quality parameters.

Preferably, the continuous determination of the number of concentrations and the provision of the measurement signals take place in a cycle time of less than 10 minutes, preferably less than 5 minutes, more preferably less than 10 seconds, even more preferably less than 5 seconds, most preferably less than one second.

In this way, the sampling rate of the gas measuring system can be adapted to the dynamic behavior of the seed germination, and the measurement signals relevant to determining the quality parameter can be obtained with sufficient temporal resolution.

Preferably, the quality parameter indicates a germination capacity and preferably also a sprouting force of the seed stock.

Preferably, the gas measuring system has a plurality of sample chambers and a corresponding plurality of electrochemical gas sensors, wherein each sample chamber with a respective electrochemical gas sensor forms a subunit of the gas measuring system so that the gas measuring system comprises a plurality of subunits for receiving a corresponding plurality of seeds of the seed stock.

In other words, in this way, a multi-sensor array can be provided.

In this way, multiple individual measurements can advantageously be carried out in parallel, so that a sample quantity for determining the quality parameter can be increased.

Preferably, the gas measuring system further has a number of sensors for monitoring a germination environment of the seed or of the plurality of seeds.

The number of sensors can be singular or plural.

It is preferred that the number of sensors for monitoring a germination environment be configured to provide information about conditions in the sample chamber(s) which affect the germination of the seed(s). This may, for example, be a humidity and/or temperature in the sample chamber(s). For this purpose, the number of sensors can, for example, be arranged in the sample chamber(s). Each of the number of sensors can be designed, for example, as a humidity sensor and/or as a temperature sensor. Additional or alternative visual monitoring of the germination conditions is also possible, for which purpose a sensor for monitoring the germination environment can be designed as a camera, for example.

It is preferred that a humidity sensor and/or a temperature sensor be arranged in each sample chamber and be connected to an or the evaluation unit via an or the energy and/or data interface.

It is possible to provide a plurality of similar aforementioned sensors in one, some, or all of the sample chambers so that a gradient of the particular property of the germination environment within each sample chamber can be monitored. For this purpose, for example, in one, some, or all of the sample chambers two temperature sensors and/or two humidity sensors can be arranged.

### BRIEF DESCRIPTION OF THE DRAWINGS

These and other features and advantages of the disclosure will become apparent from the following description of the figures. Shown are:

FIG. 1a a schematic representation of a gas measuring system according to one or more example arrangements,

FIG. 1b a schematic representation of another gas measuring system according to one or more example arrangements,

FIG. 2 a schematic representation of a variant of the gas measuring system according to one or more example arrangements, and

FIG. 3 a schematic representation of two measurement signal courses over time according to one or more example arrangements.

### DETAILED DESCRIPTION

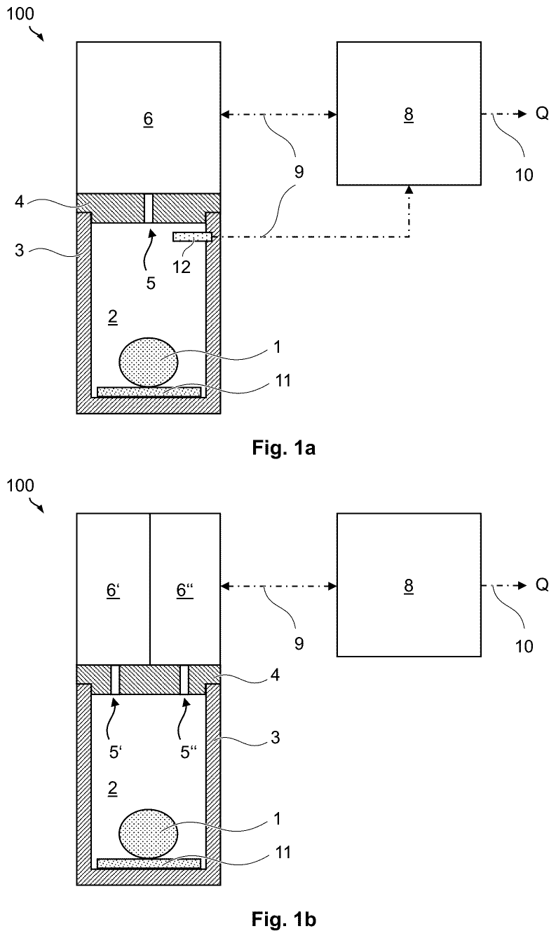

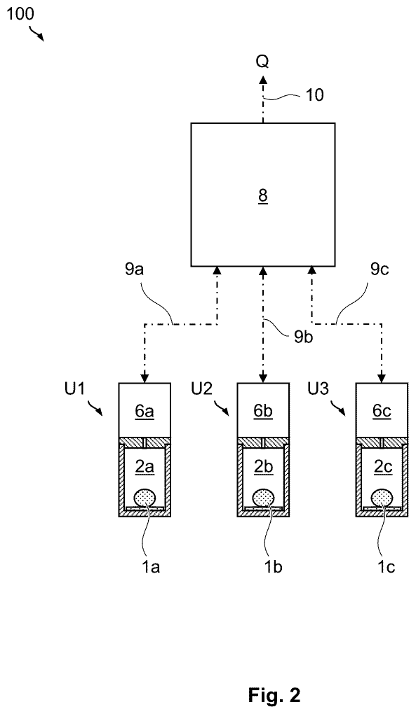

According to the disclosure, a gas measuring system 100 is provided. An example of such a gas measuring system 100 is shown in FIG. 1a. A variant of a gas measuring system 100 according to the disclosure is shown in FIG. 1b. Another variant of a gas measuring system 100 according to the disclosure is shown in FIG. 2.

Insofar as the gas measuring system 100 is referred to in general below, all variants of gas measuring systems 100 according to the disclosure are meant.

The gas measuring system 100 is used to determine a quality parameter Q of seed stock.

The gas measuring system 100 has a sample chamber 2, 2a, 2b, 2c for receiving a seed 1, 1a, 1b, 1c of the seed stock. The sample chamber 2, 2a, 2b, 2c can, for example, be designed as a substantially cylindrical or cuboid-shaped sample container 3. In addition to the seed 1, 1a, 1b, 1c, the sample chamber 2, 2a, 2b, 2c can, for example, also accommodate a fleece 11 for regulating the humidity of the sample chamber 2, 2a, 2b, 2c. The sample chamber 2, 2a, 2b, 2c can be closable by a lid 4, for example. Lid 4 and sample container 3 can be sealed against the environment or can have a predetermined gas permeability. If a lid 4 is provided, it is preferred that a through-opening 5 be formed in the lid 4, as can be seen in FIG. 1 and FIG. 2. It is also possible to form multiple through-openings 5′, 5″ in the lid 4, as can be seen in FIG. 2.

The gas measuring system 100 further has a number of electrochemical gas sensors 6, 6′, 6″, 6a, 6b, 6c, which are fluidically connected to the sample chamber 2, 2a, 2b, 2c in order to determine a number of concentrations of a number of target components of a gas present in the sample chamber 2, 2a, 2b, 2c and to provide measurement signals M, M1, M2, . . . corresponding to the number of concentrations.

If a lid 4 with a through-opening 5, 5′, 5″ is provided, it is preferred that, by means of the through-opening 5, 5′, 5″, a fluidic connection between the sample chamber 2, 2a, 2b, 2c and the number of electrochemical gas sensors 6, 6′, 6″, 6a, 6b, 6c be achieved. In the exemplary embodiments according to FIGS. 1a and 2, an electrochemical gas sensor 6 or a plurality of electrochemical gas sensors 6a, 6b, 6c are for this purpose connected to the sample chamber 2 or the sample chambers 2a, 2b, 2c by means of a through-opening 5 or a plurality of through-openings 5a, 5b, 5c. In the exemplary embodiment according to FIG. 1b, a plurality of electrochemical gas sensors 6′, 6″ are connected for this purpose to the sample chamber 2 by means of a plurality of through-openings 5′, 5″.

It is preferred that the measurement signals M, M1, M2, . . . be provided via an energy and/or data interface 9, 9a, 9b, 9c.

According to the disclosure, the determination of the number of concentrations and the provision of the measurement signals M, M1, M2 take place continuously.

The gas measuring system 100 according to the disclosure further comprises an evaluation unit 8, which is configured to receive the measurement signals M, M1, M2, . . . automatically, e.g., via the energy and/or data interface 9, 9a, 9b, 9c, to determine the quality parameter Q from the measurement signals M, M1, M2, . . . , and to provide the quality parameter Q via a data interface 10. The data interface 10 can be designed, for example, as a connection to a PC or to a display.

It is shown in the exemplary embodiment according to FIG. 1a, but possible in all exemplary embodiments, that the gas measuring system 100 can further comprise a number of sensors 12 for monitoring a germination environment of the seed 1 or of the plurality of seeds 1a, 1b, 1c. The number of sensors 12 can preferably be connected to the evaluation unit 8 via the energy and/or data interface 9, 9a, 9b, 9c. The information obtained by the number of sensors 12 about the germination environment and thus the germination conditions of the seed 1 or of the plurality of seeds 1a, 1b, 1c can be included in the determination of the quality parameter Q.

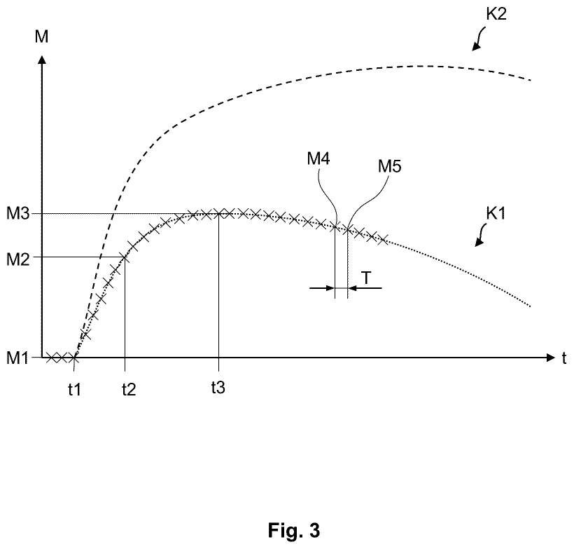

FIG. 3 shows a schematic representation of two example measurement curves K1, K2, which can be ascertained by means of the gas measuring system 100 according to the disclosure. Measurement curve K1 shows the course of the measurement signals (e.g., M, M1, M2, . . . etc.) over time t, wherein each of the continuously recorded measurement signals (e.g., M, M1, M2, . . . etc.) is indicated as a cross. Through data processing steps, for example, through interpolation, a measurement curve K1, K2 can be obtained from the measurement signals M, M1, M2, . . . . Measurement curve K1 shows a measurement signal M behavior of a good seed 1, 1a, 1b, 1c as a function of time t, while measurement curve K2 shows a measurement signal M behavior of a bad seed 1, 1a, 1b, 1c as a function of time t.

It is evident that characteristics of the examined seed 1, 1a, 1b, 1c can be obtained through the qualitative course of the corresponding measurement curve K1, K2 and through quantitative parameters of the corresponding measurement curve K1, K2, which characteristics indicate a quality parameter of the corresponding seed stock. For example, from the measurement signals (e.g., M, M1, M2, . . . etc.) directly or from the measurement curve K1, K2 obtained therefrom, a time t1 can be ascertained at which germination begins, a time t2 can be ascertained at which an inflexion point is present in the measurement signal M course over time t, and a time t3 can be ascertained at which a measurement signal M maximum is present. For example, it is also possible to integrate the obtained measurement curves K1, K2 in order to obtain a statement about a total quantity of the number of target components formed during the measurement. Depending upon which seed germination property is to be used to assess quality, the quality parameter Q can be determined from all or some of these characteristic parameters.

It is preferred that the quality parameter Q indicate a germination capacity and preferably also a sprouting force of the seed stock.

It is preferred that the number of electrochemical gas sensors 6, 6′, 6″, 6a, 6b, 6c be configured to determine a concentration of ethanol Et as a target component of the number of target components. Additionally or alternatively, it is preferred that the number of electrochemical gas sensors 6, 6′, 6″, 6a, 6b, 6c be configured to determine a concentration of oxygen and/or a concentration of carbon dioxide as target components of the number of target components.

It is further preferred that the continuous determination of the number of concentrations and the provision of the measurement signals (e.g., M, M1, M2, . . . etc.) take place in a cycle time T of less than 10 minutes, preferably less than 5 minutes, more preferably less than 10 seconds, even more preferably less than 5 seconds, most preferably less than one second. In FIG. 3, the time interval and thus the cycle time T in which the measurement signals M4 and M5 are obtained is indicated schematically. It is preferred that the cycle time T, which lies between two consecutive measurement signals (e.g., M, M1, M2, . . . , etc.) be constant.

The gas measuring system 100 according to FIG. 2 differs from the gas measuring systems according to FIGS. 1a and 1b in that a plurality of previously described sample chambers 2a, 2b, 2c and a corresponding plurality of electrochemical gas sensors 6a, 6b, 6c are provided, wherein each sample chamber 2a, 2b, 2c with a respective electrochemical gas sensor 6a, 6b, 6c forms a subunit U1, U2, U3 of the gas measuring system 100 so that the gas measuring system 100 comprises a plurality of subunits U1, U2, U3 for receiving a corresponding plurality of seeds 1a, 1b, 1c of the seed stock. A multi-sensor array gas measuring system 100 is thus formed. In this variant as well, each lid 4 can have a plurality of through-openings 5′, 5″ in order to be fluidically connected to a plurality of electrochemical gas sensors 6′, 6″ or a multisensor.

While only three sample chambers 2a, 2b, 2c and three electrochemical gas sensors 6a, 6b, 6c are shown in FIG. 3, the plurality of sample chambers 2a, 2b, 2c and the corresponding plurality of electrochemical gas sensors 6a, 6b, 6c are arbitrarily scalable.

All features described herein can be combined with one another as desired as long as this is not contradictory or relates to alternatives.

### LIST OF REFERENCE SIGNS

100 gas measuring system
1, 1a, 1b, 1c seeds
2, 2a, 2b, 2c sample chamber
3 sample container
4 lid
5,5′, 5″ through-opening
6, 6′, 6″, 6a, 6b, 6c electrochemical gas sensor
7 measurement signals
8 evaluation unit
9, 9a, 9b, 9c energy and/or data interface
10 data interface
11 fleece
12 sensor
Q quality parameter
Et ethanol
K1, K2 measurement curve
M, M1, M2, . . . measurement signal
t1, t2, t3 time
T cycle time
U1, U2, U3 subunit

1-6. (canceled)

7. A gas measuring system for determining a quality parameter of seed stock, comprising:
a sample chamber for receiving a seed of the seed stock;
one or more electrochemical gas sensors, wherein the one or more electrochemical gas sensors are fluidically connected to the sample chamber and wherein the one or more electrochemical gas sensors are configured to:
determine at least one concentration of at least one target component of a gas present in the sample chamber; and
provide a plurality of measurement signals corresponding to the at least one concentration,
wherein the determination of the at least one concentration and the providing of the plurality of measurement signals are repeated after a threshold quantity of time; and
an evaluation unit which is configured to:
receive the plurality of measurement signals automatically;
determine the quality parameter based on the plurality of measurement signals; and
provide the quality parameter via a data interface.

8. The gas measuring system of claim 7, wherein the at least one target component comprises one or more of:
a concentration of ethanol,
a concentration of oxygen, or
a concentration of carbon dioxide.

9. The gas measuring system of claim 7, wherein the threshold quantity of time is less than 10 minutes.

10. The gas measuring system of claim 7, wherein the threshold quantity of time is less than one second.

11. The gas measuring system of claim 7, wherein the quality parameter comprises a germination capacity of the seed stock.

12. The gas measuring system of claim 7, wherein the quality parameter comprises a sprouting force of the seed stock.

13. The gas measuring system of claim 7, further comprising:
one or more additional sample chambers; and
one or more additional electrochemical gas sensors corresponding to the one or more additional sample chambers,
wherein each sample chamber with a respective electrochemical gas sensor forms a subunit of the gas measuring system, wherein each subunit of the gas measuring system is configured to receive a corresponding plurality of seeds of the seed stock.

14. The gas measuring system of claim 7, further comprising:
one or more additional sensors for monitoring a germination environment of the seed.

15. The gas measuring system of claim 7, further comprising:
a fleece configured to regulate humidity of the sample chamber.

16. The gas measuring system of claim 7, wherein the evaluation unit comprises a microprocessor, and wherein the evaluation unit is configured to establish a wireless data connection between the one or more electrochemical gas sensors and the data interface.

17. A method comprising:
establishing a connection between a gas measuring device and an evaluation unit;
wherein the gas measuring device comprises:
a sample chamber for receiving a seed of a seed stock;
one or more electrochemical gas sensors, wherein the one or more electrochemical gas sensors are fluidically connected to the sample chamber;
measuring, using the one or more electrochemical gas sensors, at least one concentration of at least one target component of a gas present in the sample chamber;
providing a plurality of measurement signals corresponding to the at least one concentration;
repeating, after a threshold quantity of time, the measuring and the providing;
receiving, automatically and by the evaluation unit, the plurality of measurement signals;
determining, based on the plurality of measurement signals, a quality parameter of the seed stock; and
providing, via a data interface, the quality parameter.

18. The method of claim 17, wherein the at least one target component comprises one or more of:
a concentration of ethanol,
a concentration of oxygen, or
a concentration of carbon dioxide.

19. The method of claim 17, wherein the threshold amount of time is less than 10 minutes.

20. The method of claim 17, wherein the threshold amount of time is less than one second.

21. The method of claim 17, wherein the quality parameter comprises one or more of:
a germination capacity of the seed stock, or
a sprouting force of the seed stock.

22. A gas measuring device comprising:
a sample chamber for receiving a seed of a seed stock;
one or more electrochemical gas sensors, wherein the one or more electrochemical gas sensors are fluidically connected to the sample chamber; and
one or more additional sensors configured to monitor a germination environment of the seed,
wherein the one or more electrochemical gas sensors are configured to:
determine at least one concentration of at least one target component of a gas present in the sample chamber; and
provide, to an evaluation unit, a plurality of measurement signals corresponding to the at least one concentration,
wherein the determination of the at least once concentration and the providing of the plurality of measurement signals are repeated after a threshold amount of time.

23. The gas measuring device of claim 22, wherein the at least one target component comprises one or more of:
a concentration of ethanol,
a concentration of oxygen, or
a concentration of carbon dioxide.

24. The gas measuring device of claim 22, wherein the threshold amount of time is less than 10 minutes.

25. The gas measuring device of claim 22, wherein the threshold amount of time is less than one second.

26. The gas measuring device of claim 22, wherein the one or more electrochemical gas sensors are configured to determine a quality parameter of the seed stock, wherein the quality parameter comprises one or more of:
a germination capacity of the seed stock, or
a sprouting force of the seed stock.
